(19) United States
(12) Patent Application Publication
(10) Pub. No.: US 2010/0182415 A1
(43) Pub. Date: Jul. 22, 2010

(54) IMAGE CONTRAST ENHANCEMENT FOR IN VIVO OXYGENATION MEASUREMENTS DURING SURGERY

(76) Inventors: Eric A. Elster, Kensington, MD (US); Doug K. Tadaki, Frederick, MD (US); Nicole J. Crane, Silver Spring, MD (US); Scott W. Huffman, Cullowhee, NC (US); Ira W. Levin, Rockville, MD (US)

Correspondence Address: Naval Medical Research Center;Office of Counsel (Code 00L), 503 Robert Grant Ave., Silver Spring, MD, 20910-7500, US

(21) Appl. No.: 12/634,119

(22) Filed: Dec. 9, 2009

Related U.S. Application Data
(60) Provisional application No. 61/120,971, filed on Dec. 9, 2008.

(51) Int. Cl. A61B 6/00; H04N 7/18

(52) U.S. Cl. 348/77; 600/476; 348/E07.085

(57) ABSTRACT

A system and method for real-time or near real-time monitoring of tissue/organ oxygenation through visual assessment of contrast enhanced images of the target area of tissue or organ. Video of a target tissue/organ was acquired during surgery, selected image frames were extracted. Each extracted image is separated into red, green and blue CCD responses. A modified contrast image was created by subtracting blue CCD responses from red CCD responses, and plotting the resultant image using a modified colormap. Overlaying said modified contrast image onto the original extracted image frame under a selected transparency range, and display it for review.

### CROSS-REFERENCE OF RELATED APPLICATION

This application claims priority to U.S. Provisional Application No. 61/120,971 filed Dec. 9, 2008.

### FIELD OF INVENTION

The present invention relates generally to real-time monitoring of the oxygen levels of a target tissue area or a target organ. More specifically, the invention relates to a system and method for real-time assessment of tissue and organ oxygenation during surgery.

### BACKGROUND OF THE INVENTION

The current standard method used in assessment of organ viability during surgery is limited to visual cues and tactile feedback. However, during laparoscopic surgery, these assessment techniques are greatly impaired. A concern in laparoscopic surgery is the loss of three-dimensional assessment of organs and tissue perfusion. This is of particular relevance during laparoscopic renal donation, where the condition of the kidney must be optimized despite considerable manipulation. Currently, there is no in vivo methodology to monitor renal parenchymal oxygenation during laparoscopic surgery.

In the past 10 years, the use of living donor kidneys has markedly increased and surpassed deceased donors as the predominant source of donor organs in 2003 [1]. Laparoscopic donor nephrectomy has become a major driving force in increasing the acceptance of living donation. Laparoscopic donor nephrectomy (LDN) is thought to have several potential advantages over open donor nephrectomy (ODN) [1] [2]. Namely, laparoscopic procedures require a shorter hospital stay, decreased amounts of analgesia, allow for a faster return to work and provide improved cosmesis.

However, disadvantages of laparoscopic surgery include slightly longer warm ischemic times, and increased incidences of delayed graft function [1] [2]. Ischemia of tissue occurs when oxygen delivery to the tissue is inadequate to meet the metabolic demands. Typically, decreased blood tissue perfusion precipitates tissue hypoxia. Although, systemic hypoxia is readily diagnosed and treated, ischemic insults to individual tissue can be difficult to diagnose clinically, particularly when tissue cyanosis cannot be visually appreciated. Indolent organ injury can have a delayed impact on function. The delayed graft function thought to be the result of tissue hypoxia from pneumoperitoneum associated hypoperfusion and organ manipulation. These issues, while minor in most donors, are increasingly problematic in situations utilizing older donors, or organs intended for use in very small children [3] [4]. This is of particular concern during partial nephrectomies since organ damage results in acute renal failure in 50% of such cases [15].

Many technical aspects of laparoscopic donation have been developed to minimize organ ischemic injury, and several parameters have been monitored to indirectly assess the general tolerance of pneumoperitoneum, including cardiac output, stroke volume, mean arterial pressure, urine output, systemic vascular resistance and end-tidal CO₂. These methodologies are limited by their inability to assess the organ directly. The most direct measurement would be that of whole organ oxygenation. Unfortunately, to date there has not been a method to evaluate tissue oxygenation laparoscopically in a time frame that is clinically relevant.

The ability to intraoperatively monitor renal parenchymal oxygenation would be useful in a number of clinical situations in which prompt resolution may have a dramatic effect. For example, during the course of the operation, blood supply to the organ becomes impaired by the technical manoeuvres done during dissection (i.e., approaching the vessels from the posterior aspect). Prompt recognition of decreased oxygenation would allow for repositioning of the kidney and re-establishment of blood flow. Other examples include the determination of secondary renal arteries and the establishment of a baseline acceptable pneumoperitoneum, potentially useful in older donors.

Many systems have been developed for determining the oxygen content of blood. For example, conventional venous occlusion plethysmography has been employed for more than fifty years in muscle perfusion investigations. However, this method does not provide regional information. Ultrasound Doppler technique is another common clinical tool used to measure blood flow in large vessels, but is rather insensitive to blood flow in smaller vessels, and do not readily permit continuous measurements. Laser Doppler techniques have also been used recently to measure tissue oxygenation, but are typically limited to tissue surface. Magnetic resonance imaging (MRI) has high temporal and spatial resolution, and has become a gold standard technique in noninvasive measurement of blood flow and metabolic response, but its clinical use is limited by high cost and poor mobility.

Diffuse correlation spectroscopy (DCS) is an emerging technique use in continuous noninvasive measurement of relative blood flow in deep tissues. It has been successfully applied in studies of brain hemodynamics, PDT dosimetry and for measurement of burn depth. DCS enables measurements of relative blood flow (rBF) with high temporal and low spatial resolution in tissue. However, to date most (but not all) applications of DCS have been in small animal studies wherein source-detector separations were comparatively small. Discussion of DCS techniques has been described in U.S. Pat. No. 6,076,010.

Special techniques have also be developed to assess kidney during surgery, which include non-contact laser tissue blood flowmeter (NCLBF), pulse oximetry, fluorescein, and laser autofluorescence imaging. In a study by Ando and coworkers, NCLBF was compared with pulse oximetry and fluorescein for the assessment of ischemic tissue [10]. It was determined that NCLBF outperformed pulse oximetry and fluorescein in accuracy and sensitivity in predicting the viability of ischemic bowel. In addition, pulse oximetry and fluorescein have a high risk of failure for detecting tissue necrosis. One disadvantage of a technique like NCLBF is that the measurement is made via a pencil probe, appropriate for open surgery but not laparoscopic surgery.

Laser auto fluorescence imaging operates on the assumption that autofluorescence changes with 335 nm excitation are attributed to NADH accumulated in tissue during ischemia [11]. While the technique shows promising results and allows for real time in vivo imaging, it requires special instrumentation and is not easily converted to a format for use with a laparoscopic tower. Measurement of erythrocyte velocity using a magnifying endoscopy [12] is the only above-mentioned technique that could be easily applied during a laparoscopic surgery. However, the open lens probe of the endoscope samples and evaluates only a small portion of the tissue during a single measurement. Evaluation of the kidney as a whole would require a large number of sampling points, proving inefficient in a time limited scenario. While the OXYLITE® probe is very effective for sampling tissue oxygenation within the tissue itself [13, 14], it suffers from the same limitations as the erythrocyte velocity measurement. The needle probe allows for only spot measurements instead of global tissue oxygenation measurements.

### SUMMARY OF THE INVENTION

The present invention relates to a system and method for in vivo assessment of tissue oxygenation by acquiring and enhancing spectroscopic images of a target tissue/organ of a subject.

Furthermore, the system and method of present invention utilize standard laparoscopic equipments to provide real-time or near real-time visualization of tissue oxygenation without needing additional equipments, extra medical preparation of patients, or extensive training of the surgeon.

In one embodiment, a 3-CCD camera is used to acquire continuous video images of a target area of tissue or organ. The video images are stored. Depending on the need of the surgeon, the images may be directly displayed on a monitor or contrast enhanced to provide a visualization of the ischemic condition of the target tissue. To begin image enhancement, a selected group of image frames are extracted from the video footage and the contrast between oxygenated and deoxygenated tissue are enhanced using an image processor. More specifically, the blue CCD response is subtracted from the red CCD response, and the resultant image is plotted using a modified colormap optimized to provide the best contrast. The contrast image is then overlaid onto the original image frame under a predetermined transparency range of 40-50%, and displayed on a monitor. The surgeon thus is able to identify oxygenated tissue and organ in one color and deoxygenated tissues in a different color.

### BRIEF DESCRIPTION OF THE DRAWINGS

FIG. 1 Visible absorbance spectra of oxygenated and deoxygenated hemoglobin (HbO2 and Hb, respectively), with overlaid estimated spectra for individual charge-coupled responses (red, green, and blue, from left to right).

FIG. 2A An illustration of a color-separation beam splitter prism assembly, with a white beam entering the front, and red, green, and blue beams exiting the three focal-plane faces.

FIG. 2B A schematic drawing of a Philips type trichroic beam splitter prism. R=red, Green, and B=blue.

FIG. 3 A flow chart illustrates the general algorithm of the image contrast enhancement.

FIG. 4. Renal parenchymal oxygen tension (pO₂) measured via a fluorescence probe, as the fraction of O₂ (FiO₂) is reduced over 65 minutes. The FiO₂ is indicated with dashed arrows. At the times of blood draws, blood gas measurements were made (sO₂), and are indicated by BD.

FIG. 5. Linear relationship of calculated mean normalized ROI (region of interest) values and measured venous sO₂ as the fraction of O₂ is decreased from 100% to 8% in four different kidneys. R² is 0.9722 for kidney 1 (∘), 0.9854 for kidney 2 (□), 0.9624 for kidney 3 (Δ) and 0.9801 for kidney 4 (⋄).

FIG. 6. The normalized mean ROI values for nine donor laparoscopic nephrectomies: cases 1-9 (Figures a-i, respectively).

FIG. 7 Adsorption spectra for oxygenated hemoglobin and deoxygenated hemoglobin.

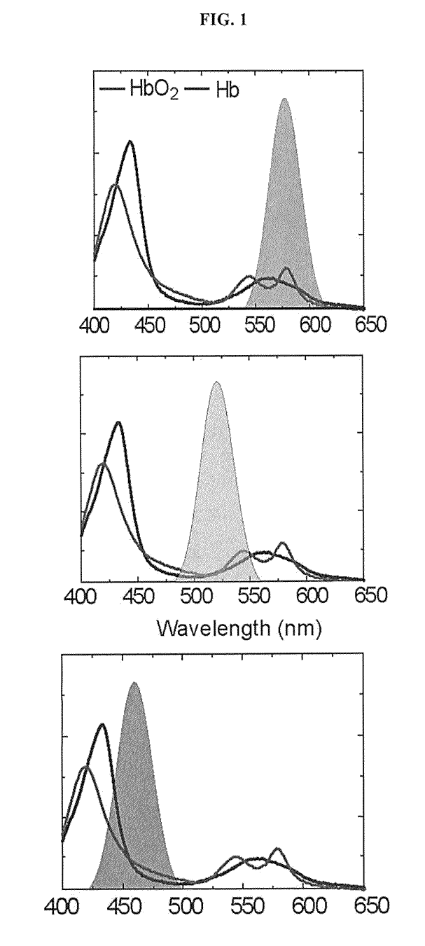

### DETAILED DESCRIPTION OF THE INVENTION

Optical reflectance spectral responses of oxygenated and deoxygenated hemoglobin in the visible region are well characterized. A component of the oxygenated hemoglobin causes a red-shift in the wavelength, which changes the spectral response from 560 nm (deoxygenated hemoglobin) to 575 nm (oxygenated hemoglobin). The present invention employs simple image processing techniques to selectively enhance this subtle difference, and thus provide the surgeon a contrast image of a target tissue/organ allowing visual assessment of a subject's tissue/organ oxygenation non-invasively during surgery in real-time or near real-time.

### Equipments

A system of the present invention comprises a) an image capturing device (100), which is capable of capturing colour images of a target area of tissue or organ; b) an image storage device (105), which is configured to store video images captured by the image capturing device (100); c) an image processor (110), which is capable of receiving input data from the image storage device, and performing image enhancement, and d) a monitor (115), which is capable of displaying image output of the image processor.

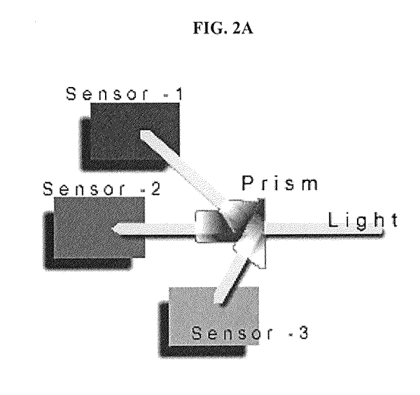

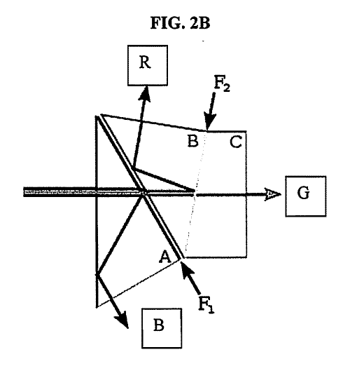

In one embodiment of the present invention, a 3-CCD camera is used to collect continuous colour images of a target tissue area or a target organ of a subject. This 3-CCD camera may be a build-in component of a standard laparoscope or a separate device operatively or wirelessly connected to the laparoscopic equipments. Three-CCD (3-CCD) is a term used to describe an imaging system employed by some still cameras, video cameras, telecine and camcorders. Three-CCD cameras have three separate charge-coupled devices (CCDs), each one taking a separate measurement of red, green, and blue light. Light coming into the lens is split by a trichroic prism assembly, which directs the appropriate wavelength ranges of light to their respective CCDs (FIGS. 2 A and B). Three-CCD cameras are often referred to as “three-chip” cameras. This term is more descriptive and inclusive, since it includes cameras that use CMOS active pixel sensors instead of CCDs, which is the definition that adopted in this application.

Three-CCD cameras are generally regarded to provide superior image quality to cameras with only a single CCD. By taking a separate reading of red, green, and blue values for each pixel, three-CCD cameras achieve much better precision than single-CCD cameras. Almost all single-CCD cameras use a Bayer filter, which allows them to detect only one-third of the color information for each pixel. The other two-thirds must be interpolated with a demosaicing algorithm to fill in the gaps.

In another embodiment, a single-CCD camera may be used as the image capture device for this invention. Instead of using narrow bandwidth filters, each image frame captured may be digitally separated into the red, green and blue color planes using standard algorithm provided by commercial software such as MATLAB® by MATHWORKS™ (Natick, Mass.).

The image storage (105) device may be any storage devices, such as a computer hard drive, a digital camcorder or a DVD recorder. This image storage device is configured to receive and store image data from said image capturing device (100), and provide digital output to an image processor. In most settings, the image storage device is operatively connected to the image capture device either as an integrated component of the image capturing device, or a storage device externally connected to the image capturing device.

The image processor (110), is equipped with image acquisition and processing algorithms, and is configured to receive data from the image storage device (105) for image processing. Processed image signal is outputted to a monitor (115) for display. The image processor is operatively connected to the monitor, as an integrated component, or as an externally connected device, for example, a microprocessor or a computer CPU.

A monitor is connected to the image processor, and is configured to receive and display the image signal feed from the image processor. Examples of a monitor may include a cathode ray tube (CRT) display, a Plasma Display Panel (PDP) display, a liquid crystal display (LCD), a Digital Light Processing (DLP) display, a Liquid crystal on silicon (LCos), a surface-conduction electron-emitter display (SED), a field emission display technology (FED) display, or a Organic Light Emitting Diode (OLED) display.

Data between above mentioned devices may also be transmitted wirelessly, and may be compressed and decompressed for transmission.

### Image Processing Algorithms

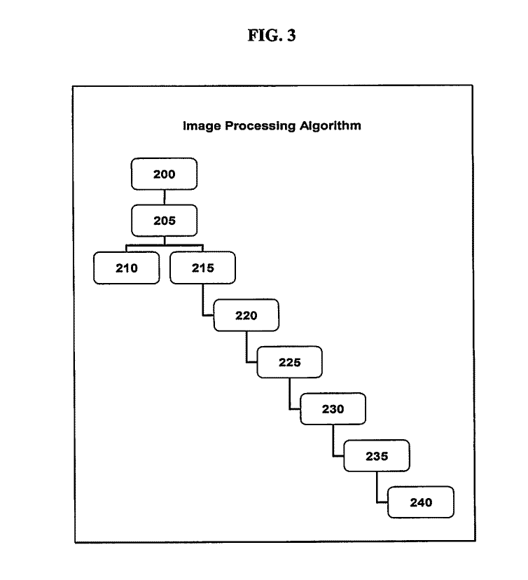

As shown in FIG. 3, when video images (200) of a target area of tissue or an organ of a subject is captured during surgery by an image capturing device, it is first stored in an image storage device, which may involve standard image compression and decompression, and image normalization (205). Based on the surgeon's need, videos may be displayed directly on a monitor (210) or contrast enhanced (215-235) to reveal oxygenation status of a target area of tissue or organ. If the surgeon chooses to assess tissue oxygenation of a target area, all or a selected group of image frames may be extracted from the video footage (215). In one embodiment, each image is extracted as uncompressed TIFF (tagged image format file) file. Each extracted image is then separated into three CCD responses: red, blue and green (220). The blue CCD response (plane) is then subtracted from the red CCD response (plane) generating a contrast image of the target area (225). This contrast image is plotted based on intensity using a modified colormap designed to provide a clear designation of red and blue values with appropriate gradation of middle values (230), which enables the preservation of anatomical details as well as a clear delineation of a mean intensity. The enhanced image is then overlaid onto the original extracted image using a predetermined transparency factor, thus creating an overlaid image (235), which is then displayed on the monitor (240). In one embodiment, a transparency range of 45-55% has been selected based on manual examination of hundreds of overlaid images from real porcine and human nephrectomy footages. Overlaid images with 35% transparency obscured some anatomical details of the image. A transparency of 65% did not provide enough contrast in some of the images. Transparency range of 40-55% has been employed for the validation testing of the present invention, but may be chosen on a case to case basis. In an embodiment, the surgeon may have the option to adjust the transparency range for each surgery depending on the anatomical details and contrast level displayed under the standard operation lighting.

A similar system for real-time visualization of tissue ischemia is described in U.S. Pat. No. 6,083,158. This real-time display of tissue ischemia comprises three CCD video cameras, each with a narrow bandwidth filter at the correct wavelength, all around 550-570 nm. The cameras simultaneously view an area of tissue suspected of having ischemic areas through beamsplitters. The output from each camera is adjusted to give the correct signal intensity for combining with the others into an image for display. Measurement at three wavelengths, combined into a real-time Red-Green-Blue (RGB) video display with a digital signal processing (DSP) board to implement image algorithms, provides direct visualization of ischemic areas.

The present invention differs significantly from the '158 patent in that no additional narrowband filters are employed, so hemoglobin responses around 420 nm are also taken into account. In addition, although a 3-CCD camera may be used in the present invention, only two of the channels are required to perform the image enhancement. Because only one CCD camera is used, there is no need to correlate and combine image responses from separate CCD cameras.

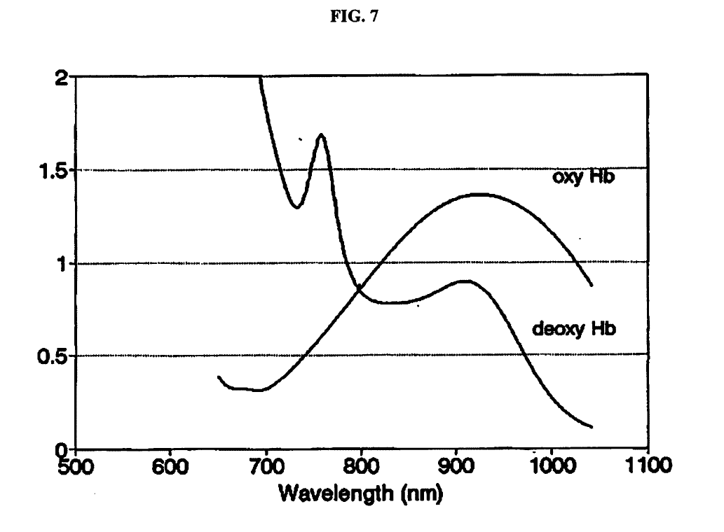

As shown in FIG. 7, deoxygenated hemoglobin shows another peak in the near-infrared region of the spectrum. In another embodiment of this invention, near Infrared responses around 760 nm may also be sampled to create an additional IR imagemap, which may be used to enhance intensity of the enhanced image.

### Validation Using a Porcine Model

### Methods and Materials

Four porcine laparoscopies were carried out to validate the sensitivity of the inventive system and method. Image acquired from these experiments were used to correlate the 3-CCD mean intensity values with actual blood and tissue oxygenation of the subject.

The porcine nephrectomies were recorded using an OLYMPUS® laparoscopic tower (Orangeburg, N.Y., USA) coupled to a STRYKER® 3-CCD camera (San Jose, Calif., USA) without the laparoscope attachment. The 3-CCD camera and the tower light were mounted to an overhead operating room light such that both kidneys were evenly illuminated and in the field of view.

The recorded video was transferred to a personal computer in which individual frames were extracted as uncompressed TIFF (tagged image format file) files using ADOBE® PREMIER 6.0. All TIFF images were automatically normalized by white balance corrections performed within the laparoscopic camera's software. The intensity of each TIFF image is of the same scale, ranging from 0-255.

Using MATLAB® software (Natick, Mass., USA), the blue CCD response of each image was subtracted from the red CCD response [6]. This difference has been directly correlated with the spectral response of hemoglobin in both the blue and red regions of the visible spectrum [6]. The resulting contrast image is plotted in a colour scale using a modified colormap. An intense red colour indicates pixels receive the most intensity from the red CCD and an intense blue colour indicates pixels receiving the least amount of intensity from the red CCD. Finally, this modified contrast image is overlaid onto the original TIFF image with 50% image transparency, allowing complete visual registry along with enhancement.

In the experiment, ischemic injury was incurred by reduced fractions of inspired oxygenation (FiO₂) during surgery. The fraction of inspired oxygenation was decreased incrementally (˜100%, ˜50%, ˜30%, ˜20%, 9%) during the determination. After each decrease in FiO₂, the kidney was allowed to equilibrate for approximately 15 minutes. Standard open surgical techniques were employed to expose the kidney and renal hilum.

While the equipment employed in this study is used in an open fashion, it is designed for laparoscopic incorporation. Open porcine model was chosen so that pneumoperitoneum would not be a variable when considering the effect of blood oxygenation on the mean ROI values calculated from the 3-CCD camera. An obvious method for altered tissue oxygenation would have been clamping the renal hilum. However, it has been extremely difficult to partially clamp the hilum in a controlled fashion (progressive hilar clamping) or to allow the kidney to reperfuse in a controlled and partial manner. Without being able to control the tissue oxygenation or deoxygenation, we could not reliably collect enough data points for a clear correlation. Thus, the decreasing FiO₂ model was chosen for the validation experiment. This method directly enabled correlation of oxygen delivery with 3-CCD mean ROI values.

Video images of the exposed kidney during surgery were collected using the 3-CCD camera and compared to measured arterial and venous oxygen saturation values (saO₂ and svO₂). Mean values were calculated from the mean regions of interest (ROIs) as defined by the user. For each different FiO₂ level, mean values for the ROIs in the images were calculated from rectangle ROI comprised of 900-4,500 pixels. While ROI sizes varied for each image, the dimensions of a rectangle at a particular location in the image remained relatively consistent from image to image. The orientation of the kidney changed slightly throughout the determination.

Renal oxygen tension (pO₂) was measured directly by an OXYLITE™ fluorescence needle probe (Oxford Optronics Ltd., Oxford, UK) in the superior pole of the kidneys. Fluorescence measurements confirm changes in blood oxygen saturation in the kidney itself as a result of reduced FiO₂. Blood was also drawn from the aorta and renal veins following each equilibrium, and immediately analyzed for saO₂ and svO₂ using a portable blood gas analyzer (iStat, Abbott™ Point of Care Inc., East Windsor, N.J., USA).

### Statistical Method

Student's t-test was used to determine significant differences between ROI mean values. Means were considered significantly different with p-values less than 0.05. For comparisons of mean ROI values determined during the porcine nephrectomies, a paired t-test for sample means was applied.

### Results

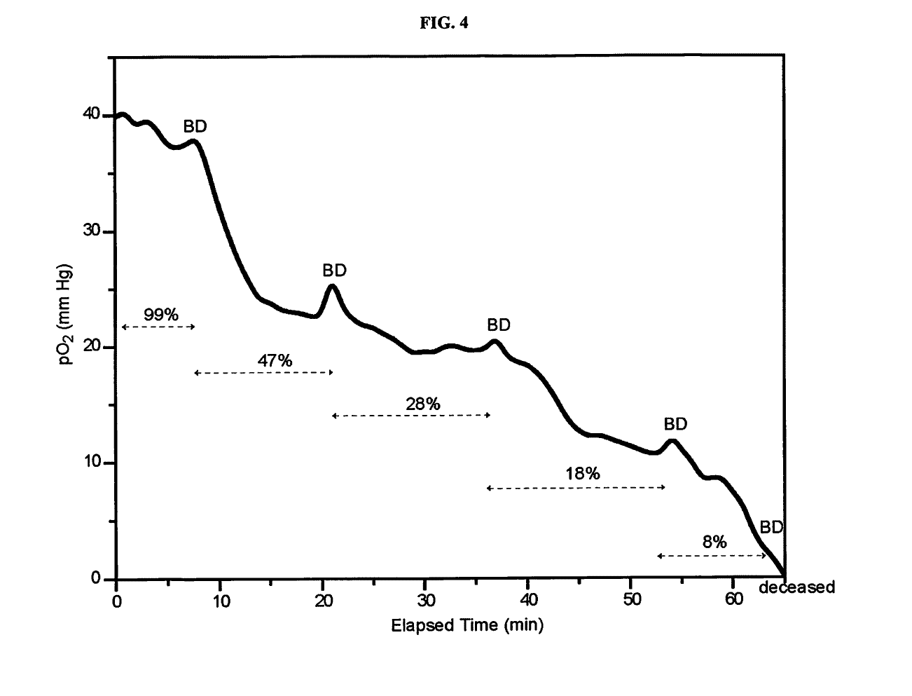

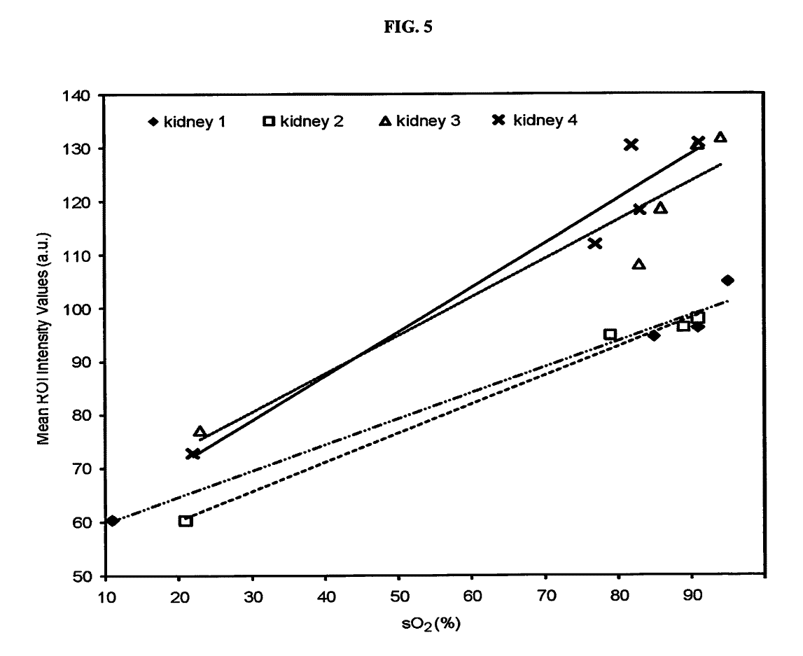

The described techniques were first tested and validated using porcine model. A fluorescent needle probe (OXYLITE™, Oxford Optronix Ltd, UK) was used to monitor changes in renal oxygenation, alongside 3-CCD assessment. In FIG. 4, the solid line follows pO₂ levels as the FiO₂ level decreased (FiO₂ is indicated over the duration of the dashed arrows). FIG. 4 demonstrates a drop in the pO₂ in the kidney each time the percentage of inspired oxygen is decreased, followed by a region of little change (equilibration). Each venous blood draw (BD) is marked by a small increase in pO₂; as the blood is drawn from the renal vein, fresh blood flows from the renal artery into the kidney, creating a temporary increase in tissue oxygenation.

At 100% inspired oxygen, saO₂ (arterial oxygen saturation value) is 100% with mean ROI values from 3-CCD modified contrast image is 0.66±0.02, 0.68±0.03, 0.60±0.03, and 0.51±0.05, for kidneys 1 through 4. Similar mean ROI values were observed for ˜50% FiO₂ with 100% saO₂ (0.70±0.01, 0.71±0.03, 0.57±0.02, and 0.55±0.06, for kidneys 1 through 4). For kidneys 1 and 2, at 28% FiO₂ and an saO₂ of 98%, the mean ROI values calculated were 0.70±0.02 and 0.69±0.02, still largely unchanged from 100 and 50% FiO₂. Similar values were observed for kidneys 3 and 4 at 21% FiO₂ and saO₂ of 91% (0.57±0.01 and 0.52±0.06). Though the drop in FiO₂ from ˜50% to 28% and 21% is significant, FiO₂'s of 28% and 21% are similar to room air, a large drop in mean ROI values from ˜50% FiO₂ to 28% and 21% FiO₂ is not expected. There is, however, a definite decrease in the mean ROI values observed for kidneys 1 and 2 with FiO₂ of 18% and saO₂ of 83% (0.64±0.03 for both kidneys). The mean ROI values drop significantly when the FiO₂ is decreased to ˜9% (0.43±0.03, 0.44±0.03, 0.36±0.02, and 0.32±0.03, for kidneys 1-4) when compared to previous mean ROI values (p=0.005). When the calculated ROI values are plotted against the saO₂ for each kidney, there is a clear linear relationship between ROI values and saO₂ measurements, as indicated by FIG. 5.

### Validation Using Human Laparoscopic Donor Nephrectomy (LDN) Model

### Methods and Materials

Healthy renal donors (n=9) were enrolled in a National Institutes of Health Institutional Review Board (NIH IRB) approved protocol to assess outcomes during and after living donor nephrectomy as well as one of several NIH IRB approved protocols to assess allograft function. LDN was performed using previously described techniques with a continuous pneumoperitoneum of 15 mmHg [7]. Renal allografts were then transplanted using standard surgical techniques. All nine kidneys were left kidneys, with a single artery, vein, and ureter, which were immediately flushed with cold University of Wisconsin solution prior to transplantation. Donor and recipient demographics are outlined in Table 1.

TABLE 1
Patient Demographics

| | Donor | BMI | | OR time | EBL | Fluid | Urine |
|---|---|---|---|---|---|---|---|
| Case | Age (yrs) | (Kg/M²) | Gender | (min) | (ml) | (ml)* | (ml) |
| 1 | 48 | 26.01 | female | 300 | 700 | 8600 | 1400 |
| 2 | 49 | | Female | 240 | 400 | 6200 | 3000 |
| 3 | 27 | 23.20 | Female | 220 | 100 | 6000 | 1400 |
| 4 | 53 | 26.23 | Male | 300 | 300 | 6600 | 1900 |
| 5 | 39 | 26.79 | Male | 240 | 400 | 8500 | 2400 |
| 6 | 26 | 23.30 | Male | 340 | <50 | 4400 | 1100 |
| 7 | 42 | 37.08 | Female | 280 | 150 | 3700 | 900 |
| 8 | 28 | 22.74 | Female | 355 | 200 | 5800 | 1400 |
| 9 | 22 | 20.69 | Male | 385 | 100 | 5800 | 1665 |
| mean | 37.1 ± 11.6 | 25.76 ± 5.03 | | 296 ± 156 | 296 ± 204 | 6178 ± 1662 | 1685 ± 660 |

BMI: body mass index; OR: operating room; EBL: estimated body loss
*Fluid was lactated riggers in all cases

Human nephrectomies were recorded using an STORZ® laparoscopic tower (Tuttlingen, Germany) coupled to a 3-CCD camera. The recorded video was transferred to a personal computer in which individual frames were extracted as uncompressed TIFF (tagged image format file) files using ADOBE® PREMIER 6.0. All TIFF images were automatically normalized by white balance corrections performed within the laparoscopic camera's software. The intensities of each TIFF image are of the same scale, ranging from 0-255.

Using MATLAB® software (Natick, Mass., USA), the blue CCD response of each image was subtracted from the red CCD response [6]. This difference has been directly correlated with the spectral response of hemoglobin in both the blue and red regions of the visible spectrum [6]. The resulting contrast image is plotted in a modified colour scale. An intense red colour indicates pixels receive the most intensity from the red CCD and an intense blue colour indicates pixels receiving the least amount intensity from the red CCD. Finally, the modified contrast image is overlaid onto the original TIFF image with 50% image transparency, allowing complete visual registry along with enhancement.

For each case and time series of extracted frames, mean values for the ROIs in the images were calculated from rectangles containing at least 625-44,000 pixels. The sizes of each rectangle were not consistent from image to image because the orientation of the kidney was constantly changing. Glare is proved troublesome by contributing false blue regions in the subtracted images. Thus, regions of glare were neglected when calculating mean values for the ROIs prior to normalization.

### Statistics Method

Student's t-test was used to determine significant differences between ROI mean values. Means were considered significantly different with p-values less than 0.05. For comparisons of mean ROI values calculated within the same human surgical case, an unpaired, two-tailed t-test with equal variances was applied.

### Results

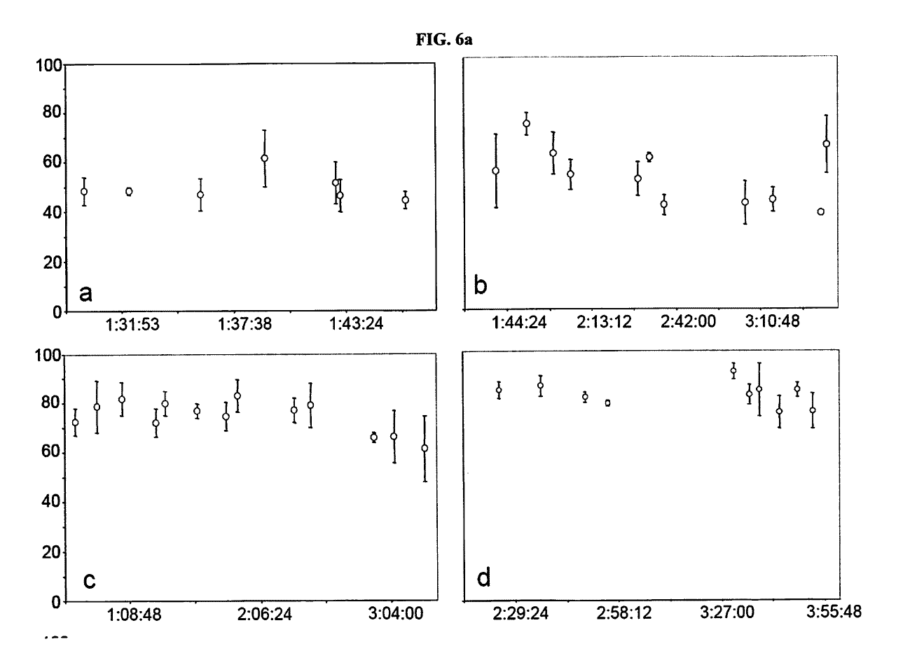

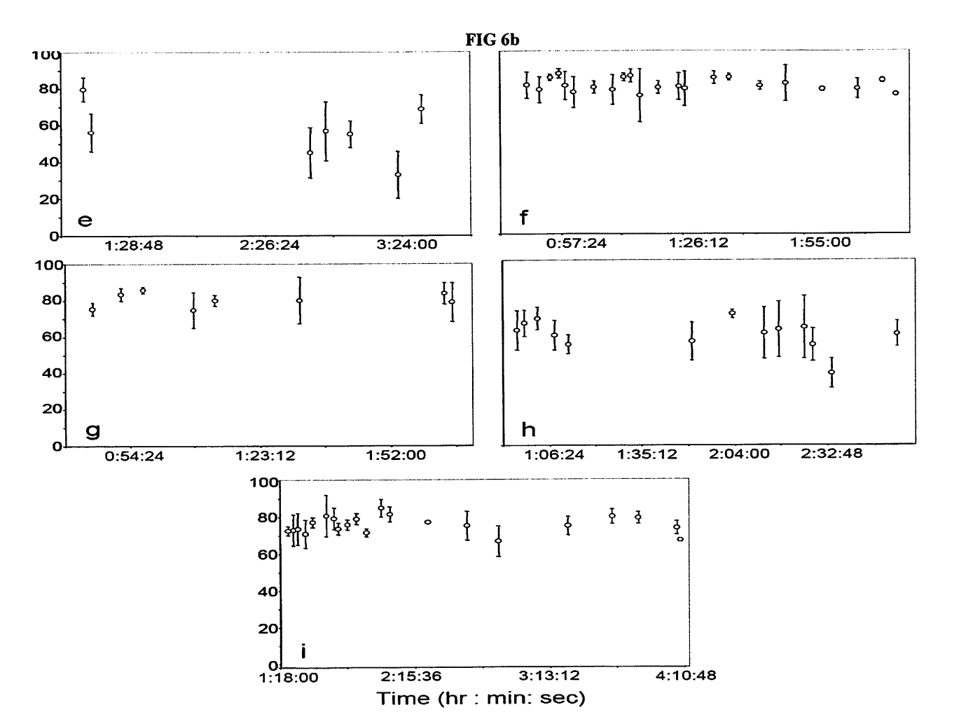

Interval monitoring of kidneys of the nine patients showed stable oxygenation without evidence of significant hypoxia. The mean values for the ROIs are presented chronologically for each case in FIG. 6. Various time points are examined, where most gaps in the sampling intervals were less than 15 minutes. For case 5, however, unambiguous image data of the kidney was not obtainable for a period of approximately 95 minutes. The duration over which image frames were collected also varied. Case 1 sampled the shortest period, with duration of approximately 16 minutes, while case 9 has the longest sampling duration, approximately 170 minutes. Cases 1 through 9 appear to fluctuate slightly with respect to ROI values but not significantly, in spite of differing normalized ROI mean intensities. The normalized ROI mean intensity values for case 5 decreased over time with respect to the starting point (79.78±6.62 versus 56.20±10.44, 44.98±13.71, 56.67±16.05, 55.07±7.24, 32.84±12.76), but returned to a comparable value by the end of the sampling period (79.78±6.62 versus 68.64±7.83).

Table 2 displays the mean starting ROI values which are compared to the mean ending ROI values for each case with the corresponding p-values. No statistically significant differences exist between the starting and the ending mean ROI values for all cases, with p-values all greater than 0.05. The mean starting ROI value for all cases, 70.21±12.36, is comparable to the mean ending ROI value for all cases, 66.43±10.53 (p=0.49). The small fluctuation in values indicates that the oxygenation of the kidney is relatively stable with an intraabdominal pressure of no more than 15 mm Hg. Note, intrapatient comparison of mean ROI values is not performed due to variability in abdominal illumination and variability of the duration of pneumoperitoneum from case to case.

TABLE 2
Mean intensity normalized ROI values

| | | | |
|---|---|---|---|
| Mean ROI | σ* | Mean ROI | σ* |

| | | | |
|---|---|---|---|
| Case | starting point | end point | p-value |

| | | | | | |
|---|---|---|---|---|---|
| 1 | 48.40 | 5.60 | 44.48 | 3.55 | 0.13 |
| 2 | 54.88 | 14.56 | 65.02 | 11.31 | 0.56 |
| 3 | 72.42 | 5.51 | 61.17 | 13.38 | 0.16 |
| 4 | 84.27 | 3.38 | 75.58 | 7.15 | 0.14 |
| 5 | 79.78 | 6.62 | 68.64 | 7.83 | 0.21 |
| 6 | 81.17 | 7.24 | 75.98 | 0.97 | 0.38 |
| 7 | 75.50 | 3.37 | 78.96 | 10.62 | 0.60 |
| 8 | 62.41 | 10.58 | 60.29 | 6.90 | 0.79 |
| 9 | 73.09 | 2.49 | 67.74 | 0.19 | 0.07 |

Mean intensity normalized ROI values of both the start and end time points for each case. All p-values are above 0.05 and indicate the mean ROI values for the start and end time points are not significantly different.
σ* is one standard devivation

Laparoscopic partial nephrectomies were also examined, where complete hilar clamping or renal arterial clamping is performed. Variation in oxygenation is observed as a direct result of surgically-induced vasoconstriction. Mean ROI values were showed to have significant decrease after clamping and then return to baseline ROI values after reperfusion (p≦0.05 in all cases).

Spectroscopic evidence for lack of change in kidney oxygenation during pneumoperitoneum is supported by standard clinical methods for assessing kidney function. Immediate graft function was seen in all recipients. The mean one day pre-operative donor serum creatinine level is 0.9±0.2 mg/dl. The mean post-operative recipient serum creatinine levels for post-operative days 1, 5 and 20 were 5.2±1.6 mg/dl, 1.6±0.4 mg/dl, and 1.5±0.4 mg/dl, indicative of brisk post transplant function (Table 3).

TABLE 3
Donor and recipient serum creatinine levels (pre- and post-operative).
Normal serum creatinine levels are ≦1.6 mg/dl.

| | Donor Serum | | Recipient Serum | |
|---|---|---|---|---|
| | Creatinine (mg/dl) | | Creatinine (mg/dl) | |
| Case | pre-op day 1 | post-op day 1 | pre-op day 5 | post-op day 10 |
| 1 | 0.8 | 5.1 | 1.5 | 1.7 |
| 2 | 0.7 | 5.1 | 1.7 | 1.6 |
| 3 | 0.7 | 7.9 | 1.8 | 1.6 |
| 4 | 0.7 | 5.6 | 1.2 | 1.0 |
| 5 | 1.2 | 4.1 | 1.1 | 0.9 |
| 6 | 1.0 | 3.6 | 1.3 | 1.3 |
| 7 | 0.8 | 4.1 | 1.4 | 1.6 |
| 8 | 0.7 | 7.9 | 2.4 | 1.7 |
| 9 | 1.1 | 3.8 | 1.9 | 2.0 |

The mean post-operative recipient BUN levels, considered normal between 8 and 20 mg/dl, (shown in Table 4) for post-operative days 1, 5 and 20 are 36±13 mg/dl, 25±8 mg/dl, and 17±5 mg/dl. With the exception of case 9, the recipient serum creatinine levels and recipient BUN levels were all within normal limits by post-operative day 20.

TABLE 4
Recipient blood urea nitrogen values at post-operative days 1, 5 and 20.
Normal BUN is 8-20 mg/dl.

| | Recipient Blood | | |
|---|---|---|---|
| | Urea Nitrogen (mg/dl) | | |
| Case | post-op day 1 | post-op day 5 | post-op day 20 |
| 1 | 30 | 27 | 15 |
| 2 | 34 | 23 | 16 |
| 3 | 53 | 35 | 20 |
| 4 | 27 | 17 | 15 |
| 5 | 58 | 21 | 12 |
| 6 | 22 | 14 | 19 |
| 7 | 35 | 20 | 15 |
| 8 | 22 | 38 | 15 |
| 9 | 45 | 27 | 28 |

### Prophetic Example 1

### Real-Time Validation Using Porcine Model

The 3-CCD (Stryker) camera and the tower light were mounted to the overhead operating room light such that both kidneys were evenly illuminated and in the field of view. The renal vein and artery were partially and sequentially clamped with Satinsky clamps until the vessels were completely occluded (i.e. immediately following 1 click, then 5 minutes post-click, immediately following a total of 2 clicks, then 5 minutes post-click, immediately following a total of 3 clicks, then 2 and 5 minutes post-click, immediately following 4 clicks, then 1, 2 and 5 minutes post-click).

At each time point of interest, an image was captured directly by the camera and processed immediately and presented as a 3-CCD contrast enhanced image. The surgeon defined the areas for ROIs and the algorithm performed and displayed a real-time calculation of the mean intensity value and its corresponding sO₂. In addition, at each time point, renal oxygen tension (pO₂) was measured directly and blood was also drawn from the aorta and renal veins following each successive clamp and immediately analyzed for saO₂ and svO₂ to validate 3-CCD contrast enhancement results.

### REFERENCES

1. Lind M Y, Ijzermans J N M, Bonjer H J: Open vs laparoscopic donor nephrectomy in renal transplantation. Br J Urol Int 2002, 89:162-168.
2. Challacombe B, Mamode N: Laparoscopic live donor nephrectomy. Nephrol Dial Transplant 2004, 19(12):2961-2964.
3. Kauvar D S, Brown B D, Braswell A W, Harnisch M: Laparoscopic Cholecystectomy in the Elderly: Increased Operative Complications and Conversions to Laparotomy. J Laparoendosc Adv Surg Tech 2005, 15(4):379-382.
4. Troppmann C, McBride M A, Baker T J, Perez R V: Laparoscopic live donor nephrectomy: A risk factor for delayed function and rejection in pediatric kidney recipients? A UNOS analysis. Am J Transplant 2005, 5(1):175-182.
5. Crane N J, Kansal N S, Dhanani N, Alemozaffar M, Kirk A D, Pinto P A, Elster E A, Huffman S W, Levin I W: Visual enhancement of laparoscopic nephrectomies using the 3-CCD camera. Proc SPIE 2006, 6081:60811-60817.
6. Crane N J, Schultz Z D, Levin I W: Contrast enhancement for in vivo visible reflectance imaging of tissue oxygenation. Appl Spectrosc 2007, 61(8):797-803.
7. Fabrizio M D, Ratner L E, Montgomery R A, Kavoussi L R: Laparoscopic live donor nephrectomy. Urol Clin North Am 1999, 26(1):247-256, xi.
8. Khauli R B, El-Hout Y, Hussein M, Dagher F J, Medawar W, Houjaji A, Sawah S, Daouk M, Uwaydah M, Abdelnoor A: A controlled sequential evaluation of laparoscopic donor nephrectomy versus open donor nephrectomy: an update. Transplant Proc 2005, 37:633-634.
9. Baldwin D D, Maynes L J, Berger K A, Desai P J, Zuppan C W, Zimmerman G J, Winkielman A M, Sterling T H, Tsai C K, Ruckle H C: Laparoscopic warm renal ischemia in the solitary porcine kidney model. Urology 2004, 64:592-597.
10. Ando M, Ito M, Nihei Z, Sugihara K: Assessment of intestinal viability using a non-contact laser tissue blood flowmeter. Am J Surg 2000, 180:176-180.
11. Fitzgerald I T, Michalopoulou A, Pivetti C D, Raman R N, Troppman C, Demos S G: Real-time assessment of in vivo renal ischemia using laser autofluorescence imaging. J Biomed Opt2005, 10(4):044018-044011.
12. Hattori R, Ono Y, Kato M, Komatsu T, Matsukawa Y, Yamamoto T: Direct visualization of cortical peritubular capillary of transplanted human kidney with reperfusion injury using a magnifying endoscopy. Transplantation 2005, 79(9):1190-1194.
13. Whitehouse T, Stotz M, Taylor V, Stidwill R, Singer M: Tissue oxygen and hemodynamics in renal medulla, cortex, and corticomedullary junction during hemorrhage-reperfusion. Am J Physiol Renal Physiol 2006, 291(3):F647-653.
14. dos Santos E A, Li L P, Ji L, Prasad P V: Early changes with diabetes in renal medullary hemodynamics as evaluated by fiberoptic probes and BOLD magnetic resonance imaging. Invest Radiol 2007, 42(3):157-162.
15. Thadhani R. Pascual M and Bonventre J V: Acute renal failure. The New England Journal of Medicine. 334:1448-1460.

What is claimed is:

1. A method for monitoring tissue oxygenation in surgery, comprising the steps of:
a. acquiring a continuous colour video of a target area of tissue;
b. extracting an image frame from said video creating an extracted image;
c. separating said extracted image into a red image response, a green image response and a blue image response;
d. subtracting said blue image response from said red image response creating a contrast image;
e. plotting said contrast image based on a modified colormap creating a modified contrast image;
f. overlaying said modified contrast image onto said extracted image based on a predetermined transparency range creating an overlaid image;
g. displaying said overlaid image; and
h. repeating steps b-g for each said extracted image.

2. The method of claim 1, wherein said image frames are extracted from said continuous colour video at a selected frequency.

3. The method of claim 1, wherein said modified colormap is optimized to provide maximum contrast of said modified contrast image and a clear view of anatomical features.

4. The method of claim 1, wherein said continuous video is acquired by a 3-CCD camera or a single-CCD camera.

5. The method of claim 1, wherein said image storage device is a computer, a flash drive, or a DVD recorder.

6. The method of claim 1, wherein said monitor is selected from group consisting of a Cathode ray tube (CRT), Plasma Display Panel (PDP), liquid crystal display (LCD), Digital Light Processing (DLP), Liquid crystal on silicon (LCos), surface-conduction electron-emitter display (SED), Field emission display technology (FED), and Organic Light Emitting Diode (OLED) display.

7. The method of claim 1, wherein steps b-h are performed upon operator command.

8. The method of claim 1, wherein said transparency range is 40%-60%.

9. The method of claim 1, wherein said method is used during laparoscopic surgery.

10. An system for monitoring tissue oxygenation, comprising
a. an image capturing device capable of acquiring a continuous colour video;
b. an image storage device capable of storing said continuous colour video;
c. an image processor capable of contrast enhancing a plurality of image frames selected from said continuous colour video; and
d. a monitor capable of displaying said contrast enhanced images from said image processor.

11. The system of claim 10, wherein said system is operatively connected to a laparoscopic station.

12. The system of claim 10, wherein said system is integrated into a laparoscopic station.

13. The system of claim 10, wherein said system is intra-connected wirelessly in part or in whole.

14. The system of claim 10, wherein said image capturing device is a 3-CCD camera or a single-CCD camera.

15. The system of claim 10, wherein said image storage device is a computer, a flash drive, or a DVD recorder.

16. The system of claim 10, wherein said image processor is a computer or a microprocessor.

17. The system of claim 10, wherein said monitor is selected from the group consisting of Cathode ray tube (CRT), Plasma Display Panel (PDP), liquid crystal display (LCD), Digital Light Processing (DLP), Liquid crystal on silicon (LCos), surface-conduction electron-emitter display (SED), Field emission display technology (FED), and Organic Light Emitting Diode (OLED) display.

18. The system of claim 10, wherein said system monitors tissue oxygenation during laparoscopic surgery.

19. The system of claim 10, wherein said contrast enhanced image represents oxygenated tissue in one colour and deoxygenated tissue in another colour.
